(19) United States
(12) Patent Application Publication
(10) Pub. No.: US 2019/0254542 A1
(43) Pub. Date: Aug. 22, 2019

(54) Methods and Apparatus for Determining Central Venous Pressure

(71) Applicant: The Regents of the University of California, Oakland, CA (US)

(72) Inventors: Rohan Wagle, Houston, TX (US); Saheel Sutaria, Albany, CA (US); Akos Toth, Cupertino, CA (US)

(21) Appl. No.: 16/400,144

(22) Filed: May 1, 2019

Related U.S. Application Data
(63) Continuation of application No. 15/303,232, filed on Oct. 11, 2016.
(60) Provisional application No. 61/980,788, filed on Apr. 17, 2014.

(51) Int. Cl. A61B 5/021; A61B 5/00; A61B 5/02; A61B 5/0402

(52) U.S. Cl. CPC A61B 5/02141; A61B 5/7285; A61B 5/6833; A61B 5/7207; A61B 5/7278; A61B 5/1102; A61B 5/0402; A61B 5/6831; A61B 5/742; A61B 5/021; A61B 5/6822; A61B 5/02028

(57) ABSTRACT

Described herein is a venous pressure monitoring system which is configured to determine central venous pressure based on jugular venous pressure. One embodiment of the JVP monitoring system may include at least one signal processor, at least one accelerometer, at least one memory 5 for storing computer instructions related to the processor(s) and/or accelerometer(s), at least one display, and at least one patch adapted to be held in place or otherwise secured to a patients neck. The signal processor may be in communication with the accelerometer(s) to translate the output from the accelerometer(s) to yield a signal and calculate the central venous pressure.

### CROSS-REFERENCE TO RELATED APPLICATIONS

This application claims the benefit of priority to U.S. Provisional Application No. 61/980,788 filed Apr. 17, 2014 which is incorporated herein by reference in its entirety.

### TECHNICAL FIELD OF THE INVENTION

The present invention relates to the field of non-invasive pressure monitors for medical purposes; more specifically, the present invention relates to a device for automatic quantification of jugular venous pressure (JVP) and estimation of central venous pressure (CVP).

### INCORPORATION BY REFERENCE

All publications and patent applications mentioned in this specification are herein incorporated by reference to the same extent as if each such individual publication or patent application were specifically and individually indicated to be so incorporated by reference.

### BACKGROUND OF THE INVENTION

The evaluation of jugular venous pressure (JVP) has been an integral part of cardiovascular examination and has important clinical diagnostic values. Jugular venous pulse pressure is produced by the changes in blood flow and pressure in central veins caused by the filling and contractions of the right atrium and right ventricle. JVP may be estimated by doing a bedside examination, which may be used for an estimation of central venous pressure (CVP). The right internal jugular vein is generally used to obtain JVP.

Based upon JVP and CVP measurements/estimations, healthcare professionals may infer information regarding hemodynamic events in the right atrium and/or diagnose diseases and abnormalities related to the heart and/or lungs. An increase in right ventricular pressure may translate to elevated jugular venous pressure, such as the case in patients with pulmonary stenosis, pulmonary hypertension, or right ventricular failure secondary to right ventricular infarction. The venous pressure may also be elevated when obstruction to right ventricular inflow occurs, such as with tricuspid stenosis or right atrial myxoma, when constrictive pericardial disease impedes right ventricular inflow, and the like. Elevated venous pressure may also result from a vena cava obstruction, and/or an increased blood volume. Some patients may have intermittently elevated venous pressure, such as patients with obstructive pulmonary disease who may have an elevated venous pressure only during expiration of breath.

The conventional technique for measuring JVP and related pressure involves examining a patient at the optimum degree of trunk elevation and then observing the venous pulsations using the naked eye. The venous pressure may be determined using a ruler to measure the vertical distance from the top of the pulsing venous column, to the level of the sternal angle, plus vertical distance to the right atrium. Because the venous pulse is generally very low amplitude and difficult to observe in some patients, this method may only provide approximate values.

The healthcare professional may measure (usually in centimeters) the vertical height of the fluid column of blood from the right atrium of the heart into the internal jugular (UI) vein by determining the highest level of the meniscus of the IJ venous pulsations. At normal atmospheric pressure, the vertical height of the fluid column (measured in cm of water) from the right atrium may be converted to millimeters of mercury (mmHg), the standard unit of measurement for CVP. A ‘normal CVP’ measurement is about 5 mmHg (or 7 cm of water). As a patient is treated for an illness, the CVP and JVP measurements for the patient may return to normal levels.

However, the manual exam by the healthcare professional to determine a CVP measurement requires the healthcare professional to be present for such an evaluation. ‘Manual exam’ is defined herein as an exam where the healthcare professional does not use any electronic device to determine CVP. The CVP manual measurements are often difficult to perform in obese patients or patients with valvular heart disease (such as tricuspid regurgitation), which results in inaccurate JVP and CVP determinations.

A more accurate method of measuring or determining JVP and/or CVP is needed. It would also be beneficial to have a device which performed automatic detection of venous pulse and/or pressure angle of the patient for calculating vertical fluid height and ultimately JVP and CVP. In addition, it would be beneficial to eliminate interference caused by the pulsing of the nearby carotid artery to increase accuracy of the device, and to clearly display the resulting JVP and/or CVP to the user.

### SUMMARY OF THE INVENTION

Described herein is a JVP monitoring system which is configured to determine central venous pressure based on jugular venous pressure. One embodiment of the JVP monitoring system may include at least one signal processor, at least one accelerometer, at least one memory for storing computer instructions related to the processor(s) and/or accelerometer(s), at least one display, and at least one patch adapted to be held in place or otherwise secured to a patient's neck. The signal processor may be in communication with the accelerometer(s) to translate the output from the accelerometer(s) to yield a signal and calculate the central venous pressure.

Generally, an apparatus for monitoring venous pressure may comprise one or more accelerometers each mounted upon one or more corresponding tabs which are positionable upon a skin of a patient and in proximity to an underlying vessel, wherein each tab is in a fixed arrangement with respect to one another and each tab is independently movable relative to an adjacent tab, a platform in communication with the one or more accelerometers, and a processor in communication with each of the one or more accelerometers, wherein the processor is programmed to monitor a position of a pulse height within the underlying vessel via movement of skin with the one or more accelerometers and determine a corresponding venous pressure.

In use, one method of determining venous pressure may generally comprise positioning one or more accelerometers mounted upon one or more corresponding tabs upon the skin of the patient and in proximity to an underlying vessel, sensing movement of the skin via the one or more accelerometers, wherein each tab is in a fixed arrangement with respect to one another and each tab is independently movable relative to an adjacent tab, determining a pulse height corresponding to the movement sensed by the one or more accelerometers via a processor in communication with each of the one or more accelerometers, and calculating venous pressure corresponding to the pulse height

In another embodiment, a non-invasive method for determining at least one central venous pressure by monitoring at least one signal with at least one accelerometer placed on a patient's neck where the accelerometer(s) is in communication with a processor. The signal(s) may correlate to pulse amplitude and/or angles of the patient at a particular position. At least one central venous pressure may be computed by processing the signal(s) received from the accelerometer(s), and the central venous pressure may be displayed.

### BRIEF DESCRIPTION OF THE DRAWINGS

The novel features of the invention are set forth. A better understanding of the features and advantages of the present invention will be obtained by reference to the following detailed description that sets forth illustrative embodiments, in which the principles of the invention are utilized, and the accompanying drawings of which:

FIG. 1 is a non-limiting simplified schematic of the JVP monitoring system;

FIG. 2 is another non-limiting simplified schematic of the JVP monitoring system;

FIG. 3 is a hardware block diagram of the JVP monitoring system in a configuration that uses two separate circuit boards;

FIG. 4 is a non-limiting hardware layout for the JVP monitoring system in a configuration having only one circuit board;

FIG. 5 is a non-limiting hardware layout for the front of a first circuit board in a configuration of the JVP monitoring system having at least two circuit boards:

FIG. 6 is a non-limiting hardware layout for the back of the first circuit board in the configuration of the JVP monitoring system having at least two circuit boards;

FIG. 7 is a non-limiting hardware layout for a second board in a configuration of the JVP monitoring system having at least two circuit boards;

FIG. 8 depicts a non-limiting JVP monitoring system placed on the neck of a patient;

FIG. 9 is a non-limiting set of software instructions for the JVP monitoring system;

FIG. 10 is a graphical analysis of the raw data received from an accelerometer; and

FIG. 11 is a graphical analysis of the same dataset from FIG. 10, where the data has been filtered.

FIG. 12 is a non-limiting embodiment of at least a portion of the JVP monitoring system.

FIG. 13 is a non-limiting embodiment of at least a portion of the JVP monitoring system.

FIG. 14 is a non-limiting embodiment of at least a portion of the JVP monitoring system.

FIG. 15 is a non-limiting embodiment of at least a portion of the JVP monitoring system.

FIG. 16 is a non-limiting embodiment of at least a portion of the JVP monitoring system.

### DETAILED DESCRIPTION OF THE INVENTION

Jugular venous pressure (JVP) can be determined by monitoring the highest position of the pulse, or the pulse height, in the internal jugular vein. Disclosed here are multiple embodiments of a JVP monitoring system and methods, at least part of which is placed on a patient's neck to monitor the pulse height in the jugular vein, and possibly other parameters, such as patient angle, using sensors. The JVP monitoring system may calculate and displays the JVP and/or CVP.

The internal jugular vein is notably different from other veins or blood vessels in the periphery of the body in that the internal jugular vein lacks valve-like structures. Peripheral veins have valves to prevent backflow of blood away from the heart and to encourage return of this venous blood to the heart. The external jugular vein, also located in the neck but anatomically lateral to the internal jugular vein, also has valves. Valves may affect manual measurement and/or observations of pulse height and cause inaccuracies in determining venous pressure. Clinicians may not be able to rely on pressure assessments from the external jugular vein because such assessments may be less accurate than an assessment or measurement from the internal jugular vein. Because the internal jugular vein lacks valves, it presents a more reliable pressure estimation from the right atrium and, thus, a more accurate assessment of central venous pressure.

Central venous pressure is the pressure at the vena cava close to the right atrium. The height, or length, of blood filled inside a jugular vein directly reflects the central venous pressure. A determination of the highest position along the jugular vein where there exists a significant pressure wave provides information to determine, measure, and/or calculate central venous blood pressure. Thus, a method of determining CVP includes determining the highest position along the jugular vein and using the resulting waveform and/or location of the waveform to calculate CVP. The “highest position” is measured from the sternal angle, which is the angle formed by the junction of the manubrium and the body of the sternum in the form of a secondary cartilaginous joint (symphysis). This is also called the manubriosternal joint or Angle of Louis. Accelerometer(s) or other sensor(s) may be used to determine the highest position, or pulse height, along the jugular vein. In the instance of only one accelerometer, or other sensor, the device may be manually moved up or down the vein until a waveform is yielded. The highest position may be determined by obtaining a waveform, then moving the portion of the JVP monitoring system which contains the accelerometers up until no waveform is obtained; the highest position is the point on the patient's blood vessel where the device still yields a waveform. Alternatively, at least two accelerometers may be used to prevent the need for manually moving the device.

The mean central venous pressure (P) may be calculated as follows:

P=5+d·sin θ

where d is the distance from the sternal angle to the highest position that yields a waveform. The addition of 5 to d represents the distance from the sternal angle to the right atrium. The symbol, θ, is the inclined angle of the upper body relative to the horizontal position.

By measuring the highest position, or pulse height, the JVP monitoring system can use this information to calculate the central venous pressure of the patient and display the result, and/or communicate the result in other ways such as wirelessly etc.

The neck is a more desirable position for applying for measuring the JVP than the forehead or other non-neck positioning on the body. In one embodiment, the sensing device of the JVP monitoring system may be placed on the neck in a position so the device is substantially directly over the internal jugular vein within the neck. A neck positioning of the device allows direct measurements of the internal jugular vein, which is a beneficial blood vessel for determining CVP/JVP. Moreover, the internal jugular vein is a fairly straight blood vessel from the heart and into the neck with minimal bending of the blood vessel, which means that the CVP or JVP determined using measurements on the neck may be more accurate than a CVP or JVP determined by a non-neck placement. ‘Neck’ is defined herein as the region between a patient's collarbone and jawbone.

Note that although the usage of the number 5 in the above equation is common practice, the number may be based on other factors such as chest size, or other factors. In the JVP monitoring system this number may be a set number, or may be calculated using an algorithm and based on other factors.

The JVP monitoring system allows for prompt recognition and monitoring of JVP fluctuations, which may be very useful for treatment of patients with conditions having a correlation to a fluctuation in JVP and/or CVP, such as but not limited to heart failure, hypervolemia, and the like.

FIG. 1 is a simplified schematic of the JVP monitoring system, including sensing device 100 and user client device 106. The sensing device 100 may have at least one accelerometer 108 therein. The device 100 may include a patch or platform 120 for placement on a patient at an external site in the vicinity of a cardiac blood vessel. The patch 120 may also include an interface between sensing device 100 and a user client device 106. In a non-limiting embodiment, the sensing device 100 may have an optional light source 110 external or internal to the device 100; however, the device 100) may sense and/or determine the central venous pressure in the absence of the light source 110. The optional light source 110 may emit light in the visible wavelength range, or any other suitable wavelength range.

The light source 110 may be any suitable light source, such as but not limited to a laser diode (e.g. RLT7605G, 760 nm, 5 mW, sm, 9.0 mmh, or RLT8510MG, 850 nm, 10 mW, sm, 5.6 mm), a light emitting diode (LED), and/or a broadband light source emitting a selected wavelength. In an embodiment, the light source may be adapted to emit light in two or more wavelengths, e.g. by association with a frequency oscillator. The light source 110 may be powered by its own power supply if the light source is exterior to the device 100, such as but not limited to a 12V DC power supply, batteries, solar power, and combinations thereof. However, if the light source is incorporated into the device 100, the light source may be powered by the same power supply as the device 100. Light from the light source 110 may be directed to at least one external tissue site on the patient that is within close proximity to a cardiac blood vessel, such as the internal jugular vein, the external jugular vein, and combinations thereof. In another non-limiting embodiment, the light source 110 may be used as an indicator of a pulse. For example, the accelerometer(s) 108 may detect a pulse of blood in the blood vessel, and the light source 100 may illuminate a pulse of light for the user which represents the blood pulse. Said differently, the light source 100 may be a type of display in the absence of or in addition to a traditional visual or audible display within the device 100.

The patch 120 may be made out of any material suitable to support the electronic components housed therein, such as the accelerometer(s) 108, optional light source 110, optional display 112, optional processor 104, optional memory 114, optional ECG sensor, etc. An example of one such suitable material for the patch 120 is medical rubber. The patch 120 may be held in position manually, may be held in position by adhesives (one side of the patch may be coated with an adhesive material, such as but not limited to a hydro gel adhesive, tape, and the like) or may be adapted to be held in place with straps that can be tied or otherwise secured to the patient's neck. Opposing ends of the patch 120 may also include an adhesive material, such as Velcro to facilitate their attachment and to hold the patch around the patient's neck.

In a non-limiting embodiment, the patch 120 may be reusable and/or recyclable. The patch 120 may have disposable adhesive layers thereon to attach the patch 120 to a first patient. After the exam of the first patient has occurred, the first disposable adhesive layer may be removed from the patch 120. A second disposable adhesive layer may be used during an exam of a second patient and so on. ‘First’ and ‘second’ are used herein to distinguish the adhesive layers, patients, etc. from one another.

The accelerometer(s) 108 may translate received venous pulse data and/or patient angle data. A non-limiting example of a suitable accelerometer 108 for use in the present device is model MMA8451Q supplied by Freescale Semiconductor. The accelerometer 108 may have computer instructions for transmitting the accelerometer data to the processor 104. The processor 104 may have computer instructions for receiving data from the accelerometer and computer instructions for forming a pulse peak viewable on the display 112, which may be viewable as a CVP number or a waveform or any other suitable format. The processor 104 may have additional computer instructions for subtracting the portion of the waveform corresponding to a carotid artery, or other interfering signals, such that the processor 104 only transmits the waveform or CVP number corresponding to the internal jugular vein to the display 112. A similar set of data may be collected from each accelerometer 108 for each pulse. The processor 104 may have computer instructions for monitoring the amplitude of each pulse measured by each accelerometer 108. The processor 104 may also have computer instructions for monitoring a change in amplitude of each pulse between pulses and/or between or among accelerometer(s) 108. The amplitude and the decay in amplitude may be used to determine the height of the jugular venous pulsation. For example, the processor 104 may determine the pulse height by the highest location where the pulse magnitude is above a certain level. The cutoff level may be preset or may be determined based on the pulse amplitude in one or more locations along the neck. Once the height of the jugular venous pulsation is determined, the accelerometers 108 may measure the angle of the patient, transmit such data to the processor 104, and the processor 104 may determine the central venous pressure.

The processor 104 may be operable to receive the signal provided by the accelerometer(s) 108 (e.g. the pulse amplitude and/or patient angle) and translate the signal into a display 112, such as a waveform. In one non-limiting embodiment, at least one CVP may be determined/calculated; however, the device 100 may determine a plurality of CVP measurements in real-time to monitor such data over a period of time. Patient treatment may vary based on the CVP measurements obtained from the device 100. ‘Real-time’ is defined herein as data that may be updated at about the same rate as received by the device 100; a non-limiting example may be where CVP data has been received by the device 100 and is displayed in five minutes or less.

Thus, the processor 104 is operable to digitize the output provided by the accelerometers) 108 into a recordable output for presenting on a display (e.g. 112). In another non-limiting embodiment, the processor 104 may be operable to receive the signal provided by the accelerometer(s) 108 (e.g. the pulse amplitude and/or patient angle) and translate the signal into a display 112, which is the final calculated value of CVP for a particular patient. In a non-limiting embodiment, the final calculated value of CVP may be presented on a ‘two digit’ display 112, i.e. a display only having two digits. However, the CVP may be presented on any type of display in the absence of or in conjunction with other values (oxygen saturation. ECG data, etc.) used during an exam where CVP would be helpful. Another non-limiting embodiment of the display 112 may be located on or include a user device (e.g. smart phone or other portable device). Thus, the processor 104 is operable to digitize the output provided by the accelerometer(s) 108 into a recordable output for presenting on a display (e.g. 112).

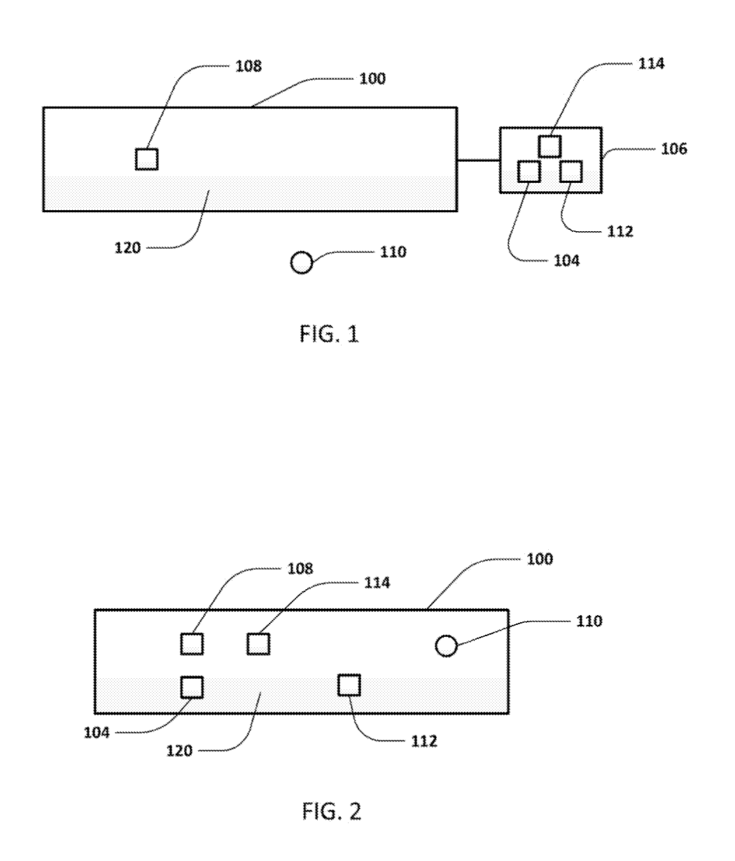

The user client device 106 may be, but is not limited to, personal computers, personal digital assistants, mobile phones, a smart phone, a tablet, or any other apparatus capable of receiving data from the accelerometer(s) 108. More than one client device 106 may receive data from the accelerometer(s). The user client device 106 may include a display 112, an optional associated memory 114, and a signal processor 104. The signal processor 104 may communicate with the accelerometer(s) 108 to translate the angle data received by the accelerometer(s) 108 into a visual display 112 to be viewed on the user client device 106. Communicating with the user client device 106 may be wireless or wired. In a non-limiting embodiment, the display 112, the memory 114, and signal processor 104 may be incorporated into the sensing device 100, instead of being external to the device 100, as depicted in FIG. 2. The user client device 106 may include one or more user input devices (not shown) such as, but not limited to, a QWERTY keyboard, a keypad, a trackwheel, a stylus, a mouse, a microphone. If the screen is touch sensitive, then the display 112 may also be used as the user input device. The display 112 may be or include, but is not limited to, an LCD screen display and/or a speaker. The data may be visually or audibly displayed. The display 112 functions in real-time to display the selected blood vessel waveform.

The computer instructions for the processor 104, accelerometer(s) 108, etc may be provided by an operating system and/or software applications located in the memory 114. Further, it is recognized that the sensing device 100 and/or the user client device 106 may include a computer readable storage medium (not shown) coupled to the processor 104 for providing instructions to the processor 104. The computer readable medium may include hardware and/or software, such as but not limited to, magnetic disks, magnetic tape, optically readable medium such as CD/DVD ROMS, and memory cards. In each case, the computer readable medium may take the form of a small disk, floppy diskette, cassette, hard disk drive, solid-state memory card or RAM. It should be noted that the above listed examples of computer readable media 212 (not shown) may be used either alone, or in combination. The memory 114 and/or computer readable medium may be used to store, for example, the desired output for use in processing the data from the accelerometer(s) 108.

Further, it is recognized that the user client device 106 may include executable applications that include software code or machine-readable instructions for implementing predetermined functions/operations including those of an operating system. The processor 104 may be a configured device and/or set of machine-readable instructions for performing operations as described. As used herein, the processor 104 may include any one or combination of, hardware, firmware, and/or software. The processor 104 may act upon information by manipulating, analyzing, modifying, converting or transmitting information for use by an executable procedure or a user client device. The processor 104 may use or comprise the capabilities of a controller or microprocessor, for example. Accordingly, the functionality of the processor 104 and/or the accelerometer 108 may be implemented in hardware, software or a combination of both. Accordingly, the use of a processor 104 as a device and/or as a set of machine-readable instructions is hereafter referred to generically as a processor/module for the sake of simplicity.

In use, the patch 120 is generally placed on the neck of the patient at a site near a selected blood vessel, for example, the internal jugular vein. It is desirable for the patient to be lying down at about a 30-degree incline from the horizontal, but any angle may be used. The patient maintains regular breathing during the process of measuring the pulse of the blood vessel. Light from the optional light source 110 may be reflected off the target site of the patient's neck to allow better visualization by the naked eye of a healthcare professional. As previously mentioned, the light source 110 may be used as an indicator of a pulse. For example, the accelerometer(s) 108 may detect a pulse of blood in the blood vessel, and the light source 110 may illuminate a pulse of light for the user in the instance. Said differently, the light source 110 may be a type of display in the absence of or in addition to a traditional visual or audible display within the device 100. The processor 104 translates the data detected by the accelerometer(s) 108 into an output signal (e.g. pulse amplitude and/or patient angle) that may be digitized for expression as a waveform of the selected blood vessel pulse.

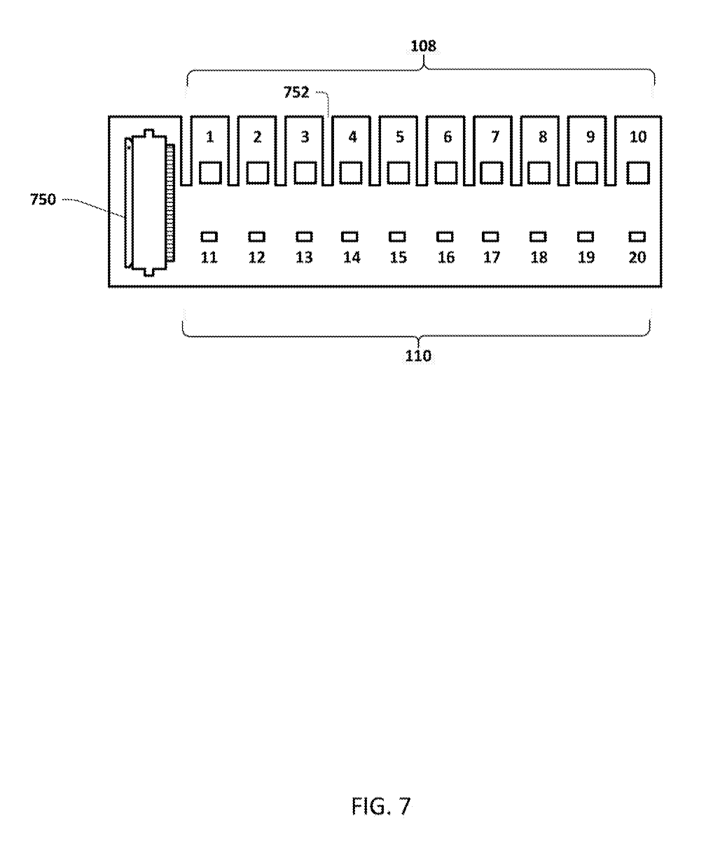

The patch 120 may be a flexible material to allow the patch 120 to conform to the contours of a patient's neck. The circuit board may have spaces and/or cutouts between each accelerometer (FIG. 7), so that each accelerometer 108 is as isolated from another accelerometer 108 as possible to precisely measure the venous pressure without interference from another accelerometer. As a pulse of blood flows through a blood vessel underneath the device 100, the surface of the skin may slightly move, and this movement may be detected by the accelerometer(s) 108. A pulse of blood having increased pressure may result in more movement at the surface of the skin, and a corresponding larger signal may be detected by the accelerometer(s) 108. Inversely, a pulse of blood having lower pressure may result in less movement at the surface of the skin, and a corresponding smaller signal may be detected by the accelerometer(s) 108.

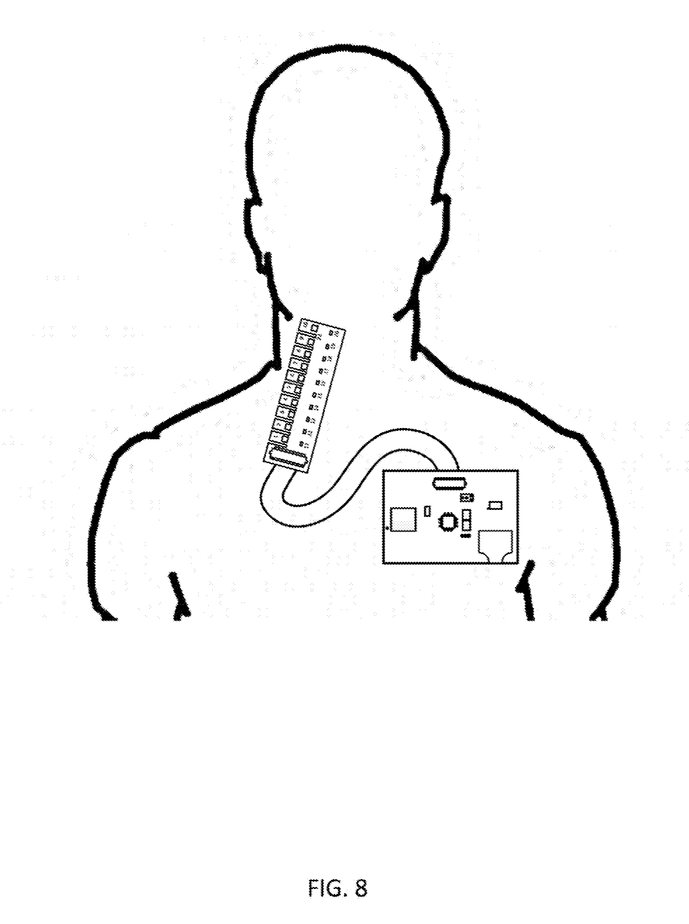

As the pulse of blood moves away from the heart, the pressure will decrease. For example, as a pulse of blood moves through the internal jugular vein from the heart towards the head, the blood may pass under each accelerometer 108 (See FIG. 8). The accelerometers 108 may detect the pulse of blood, and may detect movement at the surface of the skin from the accelerometer closest to the heart towards the accelerometer closest to the head. The movement detected by the accelerometer closest to the heart is greater than the movement detected by the accelerometer closest to the head.

FIG. 2 is a non-limiting simplified schematic of the JVP monitoring system where each component is located within the sensing device 100, such as the processor 104, the memory 114, the display 112, the optional light source 110, and accelerometers 108. However, the sensing device 100 may still be wirelessly connected or wired to another user client device.

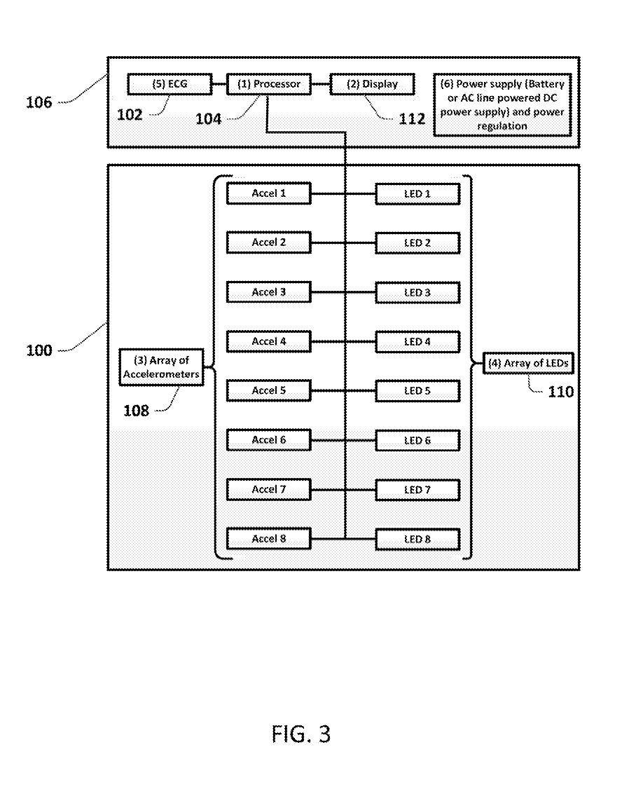

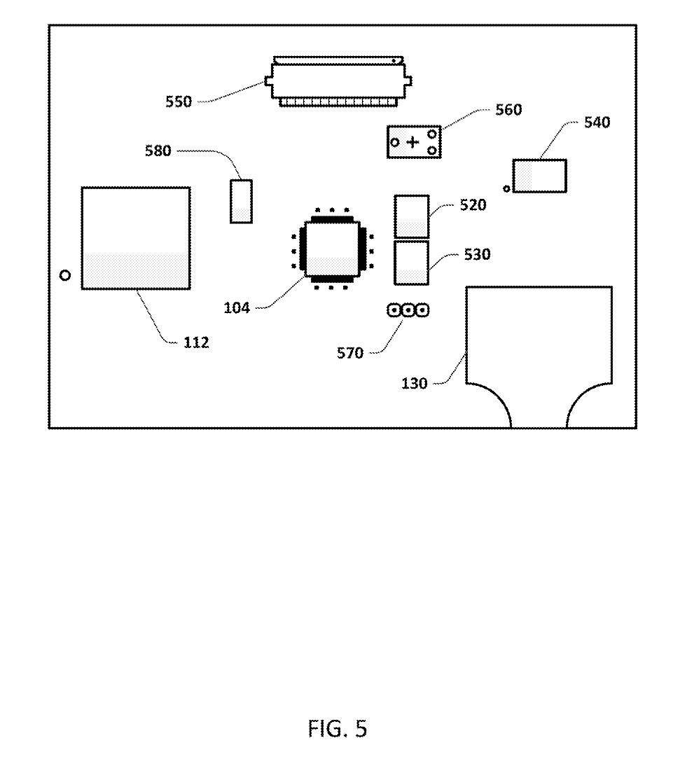

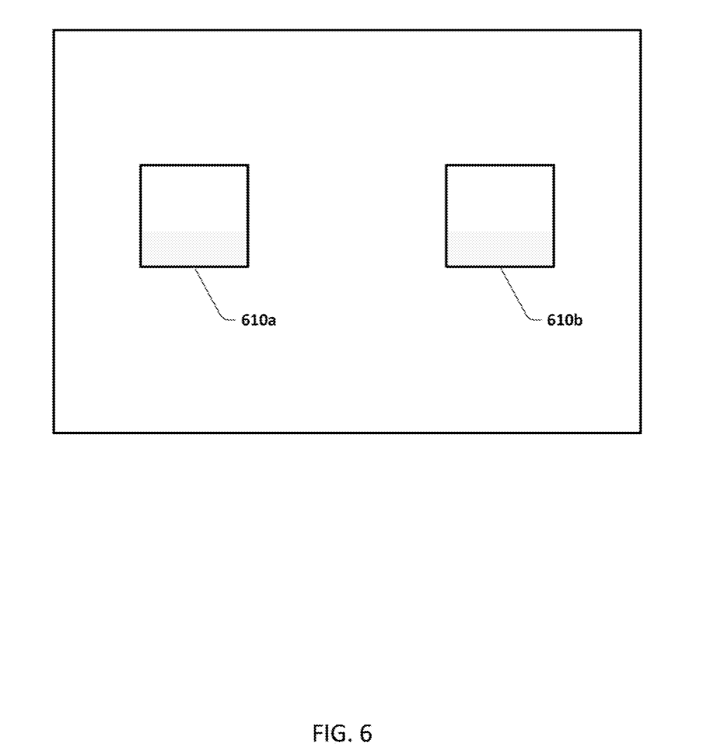

FIG. 3 depicts a non-limiting hardware configuration of the JVP monitoring system having two circuits. The main circuit board 106 may have or include a processor 104, a display 112, an optional ECG 102, and a power supply 130. The hardware layout for the first, or main, circuit board is also depicted in FIG. 5 and FIG. 6. An ECG sensor may be integrated in the device, or an ECG sensor may be external to the device. The ECG sensor 102 is optional for determination of the CVP. The ECG sensor 102 may aid the determination of CVP by gating (or synchronizing) the ECG (e.g., electrical activity of the heart) with the corresponding jugular venous pulsations. Said differently, the ECG may aid a user in identifying the carotid artery versus the jugular vein(s) from a particular waveform. For example, if the ECG and accelerometer waveforms are in sync, the corresponding peaks may be correctly identified as the jugular vein or carotid artery. Also, the synchronous use of the ECG with the accelerometer may allow for any noise or extraneous data to be subtracted, such as extraneous data related to an accidental movement by the patient. The ECG sensor 102 may include computer instructions for transmitting ECG data to the processor 104. The processor 104 may have computer instructions for receiving ECG data, further using the ECG data to determine a CVP, and transmitting the ECG data to the display 112.

The sensor circuit board 100 may have or include an array of accelerometers 108 and an optional array of light sources, such as LEDs 110.

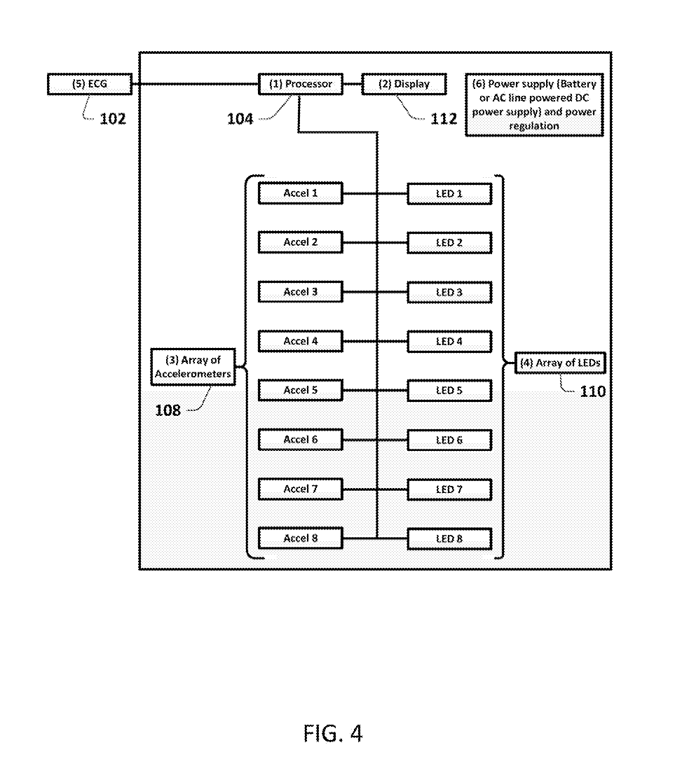

FIG. 4 is a non-limiting hardware layout of a single circuit board similar to FIG. 3, but where the main components of the main circuit board and those of the sensor circuit board are combined onto one circuit board.

FIGS. 3 and 4 depicts eight accelerometers 108 being used, however, more or fewer accelerometers may be used for such purpose depending on the power supply, size of the device, size of the patient, etc.

FIG. 5 is a non-limiting depiction of the front of the main circuit board shown in FIG. 3, which may include the processor 104, a two digit display 112, a first push button 520, a second push button 530, a power regulation circuit 540, a power supply 130, a connector 550 for connection to the sensor circuit board having the accelerometers, a programming port 560, a communication interface 570, an oscillator 580. The two-digit display may visually display the final calculated CVP and/or depict device status messages or features. Such options may be or include, but are not limited to, menus for a user to adjust brightness of the display, turn on or off the device, to store data by the pressing of a button, etc. In a non-limiting embodiment, the display 112 may be used as a user interface to accommodate such features.

The first and second push buttons (520, 530) may turn the device on/off, select an option or display format, and combinations thereof. The device may have a communication interface to send data to an external device, such as a user client device. The communication interface may also communicate with an external ECG, if desired. The power supply 130 may be any form known to those skilled in the art, such as but not limited to, a battery, a wall adapter, solar power, and the like. The power regulation circuit 540 may regulate and deliver power to the device 100. The programming port 560 may be used to add or delete computer instructions from the processor.

FIG. 6 is a non-limiting depiction of the back of the first circuit board, which includes two electrodes 610a, 610b to take an ECG measurement.

FIG. 7 is a non-limiting layout of the sensor circuit board, shown in FIG. 3, which may include the accelerometers (1-10) 108, the optional light source LEDs (11-20) 110, a connector 750 for connection to the first circuit board, and spaces or cutouts 752 in the second, or sensor, circuit board between each accelerometer. The second, or sensor, circuit board may be constructed using a flexible printed circuit board to allow the device to conform to the patient's neck, and to allow the accelerometer to move with the venous pulsation.

The array of optional LEDs may be used to visualize the pulse of blood through the internal jugular vein. The device 100 may illuminate an array of LEDs to correspond with the pulse movement detected by the accelerometers 108. When a pulse of blood travels under the array of accelerometers, a movement is detected, and a pulse of light may be illuminated from the light source 110. The LEDs may have more intensity with increased movement detected by the accelerometers, and the LEDs may have less intensity with decreased movement detected by the accelerometers. Said differently, the light source intensity directly correlates to the pulse intensity detected by the accelerometer(s) 108. The light source is not necessary for determination of the CVP. The device 100 may perform a CVP measurement in the absence of a light source.

FIG. 8 depicts a non-limiting placement of the JVP monitoring system on the neck of a patient. The main circuit board may be placed over the patient's heart for measurement by the ECG, and the sensor circuit board may be placed over the internal jugular vein to determine an estimation of a CVP. The ECG signal may be transmitted to the processor 104. The processor 104 may synchronize the ECG signal with an accelerometer signal to allow for easier identification of the carotid artery pulse and jugular venous pulse.

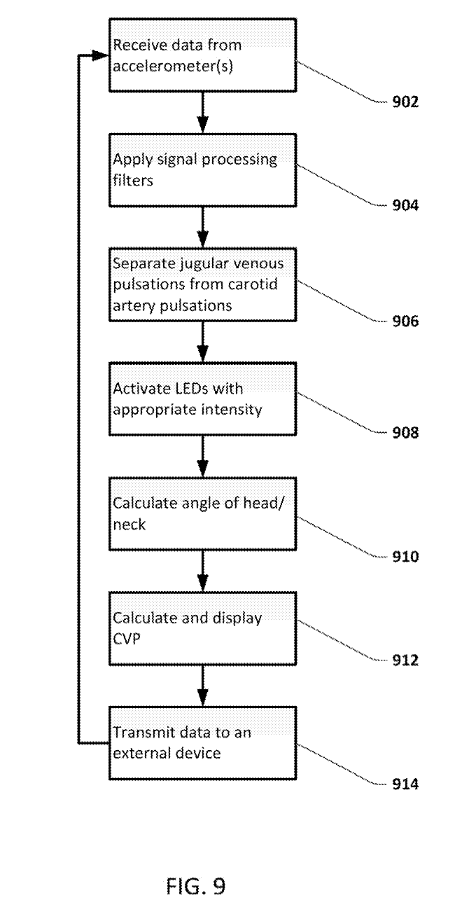

FIG. 9 is a non-limiting set of computer instructions used by the processor to determine the CVP. Box 902 represents receiving data from the one or more accelerometer(s). The received data is then processed using the appropriate signal processing filters to eliminate signal noise from the carotid artery, movement, and other sources, represented by box 904. Signal filtering may also or alternatively be performed to measure the propagation of a signal along the blood vessel. Box 908 represents the activation of the LEDs with an intensity that is correlated to the signal of the accelerometer. The LED may activate either before or after the filtering process 904. The processor then determines the angle of the head/neck, and calculates and displays the CVP. These steps are represented by boxes 910 and 912. Data is then optionally transmitted to an external device, represented by box 914. This process is repeated for as long as CVP determinations are required.

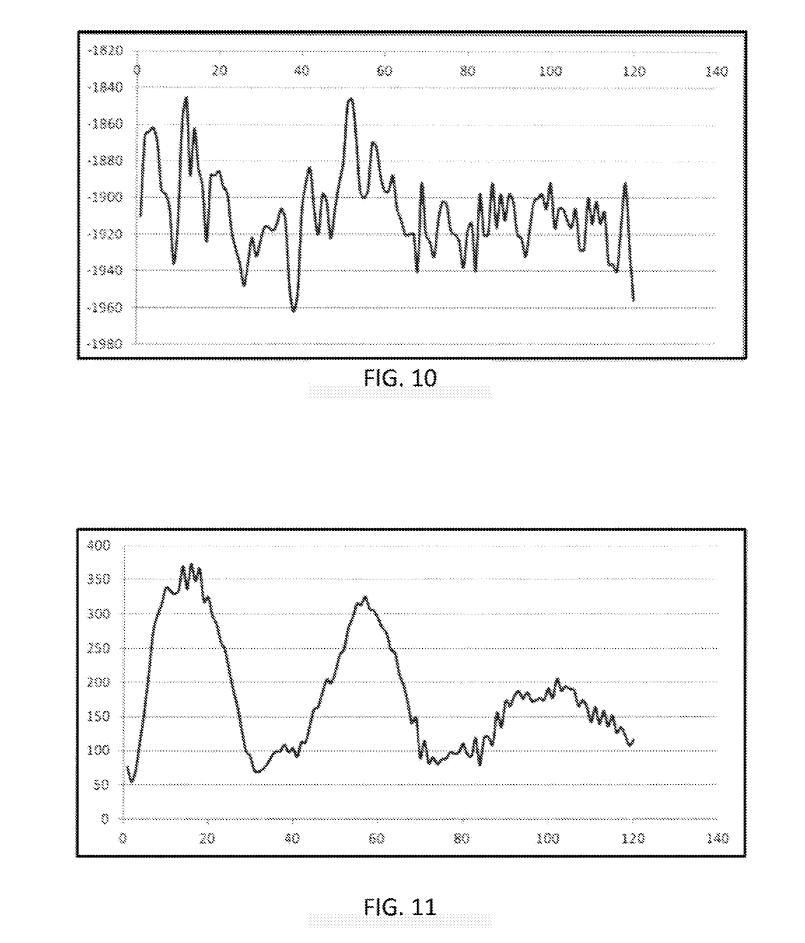

FIG. 10 is a graphical analysis of the raw data received from an accelerometer, and FIG. 11 is a graphical analysis of the same dataset from FIG. 10, after the data has been filtered and processed. Similar waveforms may be received from each accelerometer. An example of raw data received from an accelerometer for one pulse is shown in FIG. 10. An example of the same set of data after filtering is shown in FIG. 11. Although this data was collected over one pulse, three distinct peaks can be seen. Multiple peaks may be seen for each pulse because the carotid artery and the internal jugular are anatomically very close to each other. The first peak may correspond to the carotid pulse, and the next two peaks may correspond to the internal jugular vein. A combination of hardware and software filters may be used to remove noise and isolate the movement of the neck, so that only the jugular vein pressure and/or pulse may be represented by a peak.

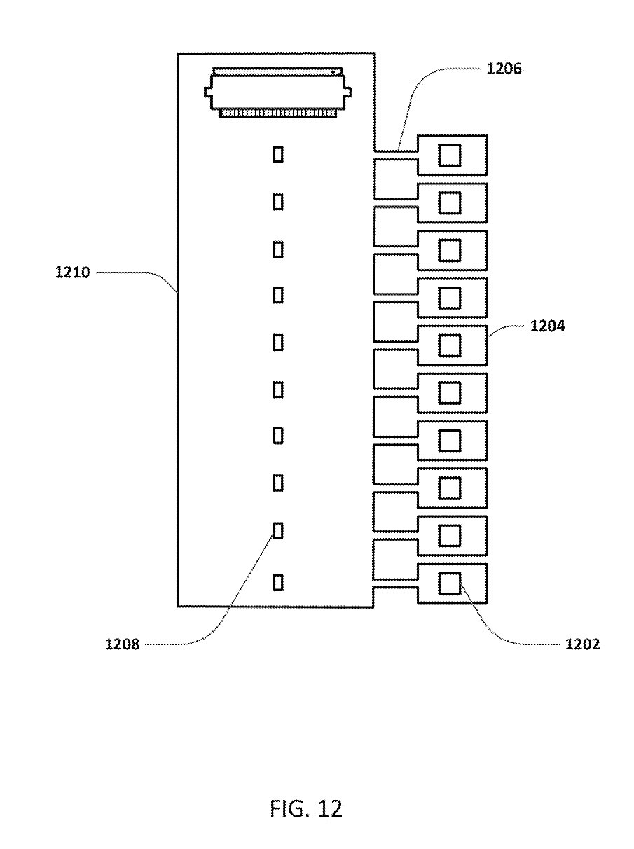

FIG. 12 is a non-limiting embodiment of at least a portion of the JVP monitoring system. In this embodiment, accelerometers 1202 are on individual tabs 1204. The tabs are separated from circuit board 1210 and indicator lights 1208 via flexible necks 1206. It is important that each accelerometer function independently, with minimal influence from the motion of the accelerometers near it. The flexibility of flexible necks 1206 allow each accelerometer to move independently of the accelerometers near it. In this way, the data collected from each accelerometer is indicative of skin movement in only that specific area. To achieve accelerometer independence in this embodiment each accelerometer is on a separate tab, connected to the circuit board via a flexible neck. The neck is flexible enough so that each tab, and therefore each accelerometer, can move independently of the other accelerometers. The tabs and flexible necks may be made out of the same material, such as a thin flexible polymer, or silicone or any other suitable material. The tab may have adhesive on the underside where the neck may not. Alternatively, both the tab and neck may have adhesive. Any circuitry necessary for the accelerometer to communicate with the circuit board may be embedded and/or attached to the tab and neck. This communication may alternatively be wireless. Neck length may be less than 1 cm. Alternatively, neck length may be 1-3 cm. Alternatively, neck length may be greater than 3 cm.

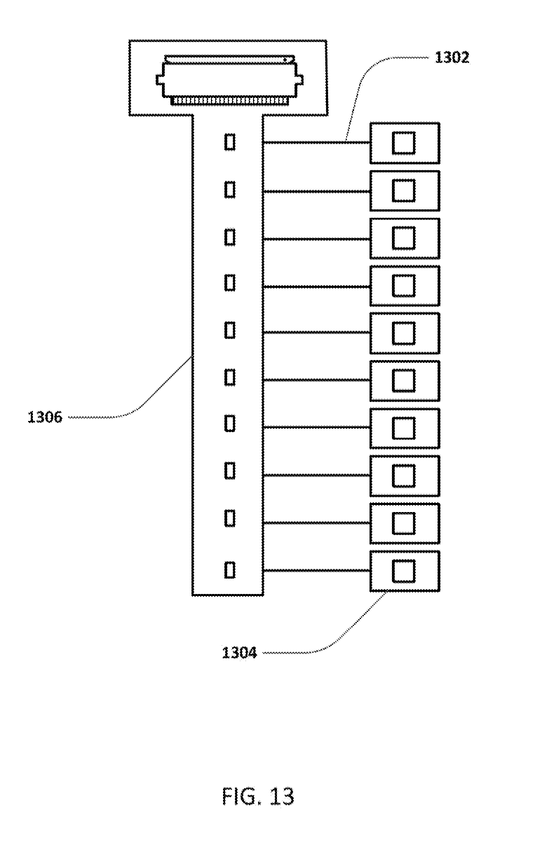

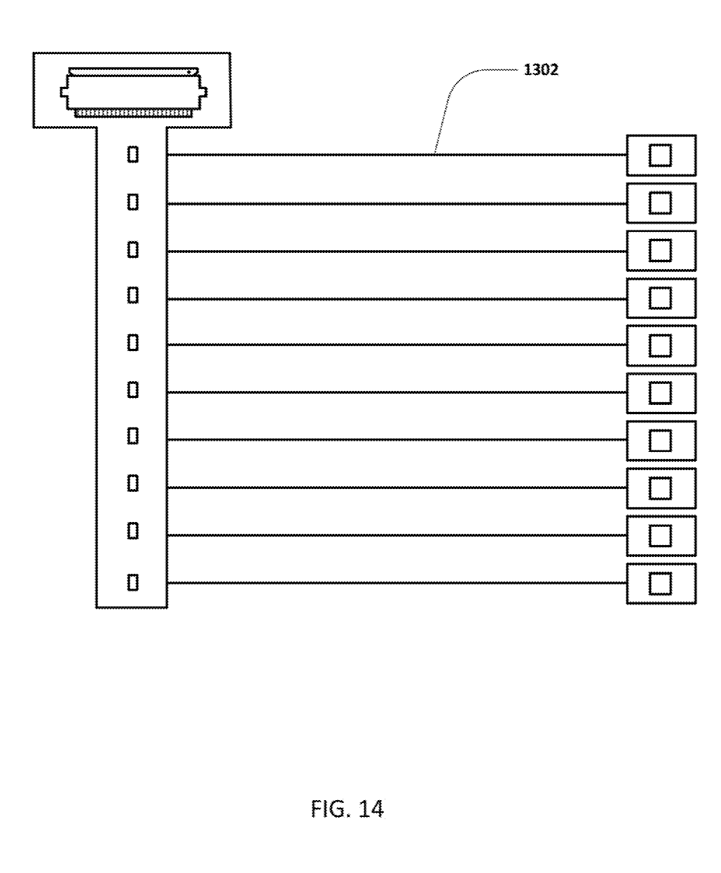

FIG. 13 is a non-limiting embodiment of at least a portion of the JVP monitoring system. In this embodiment, wires 1302 are used to connect tabs 1304 to circuit board 1306. Wires 1302 function similarly to flexible necks 1206 in FIG. 12. Using wires instead of necks may allow tabs 1304 to be placed further from circuit board 1306. The wires may serve both as communication with the circuit board and also as a physical connector with the circuit board. FIG. 14 shows an example of this embodiment where wires 1302 are longer. Wire length may be less than 1 cm. Alternatively, wire length may be 1-3 cm. Alternatively, wire length may be 3-10 cm. Alternatively, wire length may be greater than 10 cm.

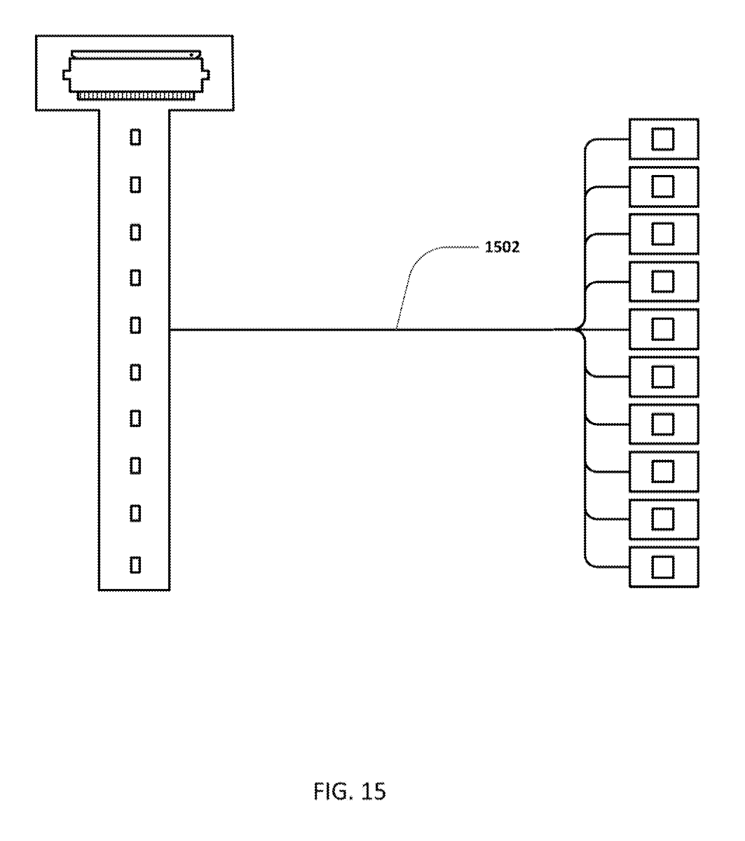

FIG. 15 is a non-limiting embodiment of at least a portion of the JVP monitoring system. In this embodiment, wires 1302 feed into a single communication wire or wire bundle 1502. This embodiment allows the accelerometers to be fairly far from the circuit board without multiple long wire connectors which may tangle.

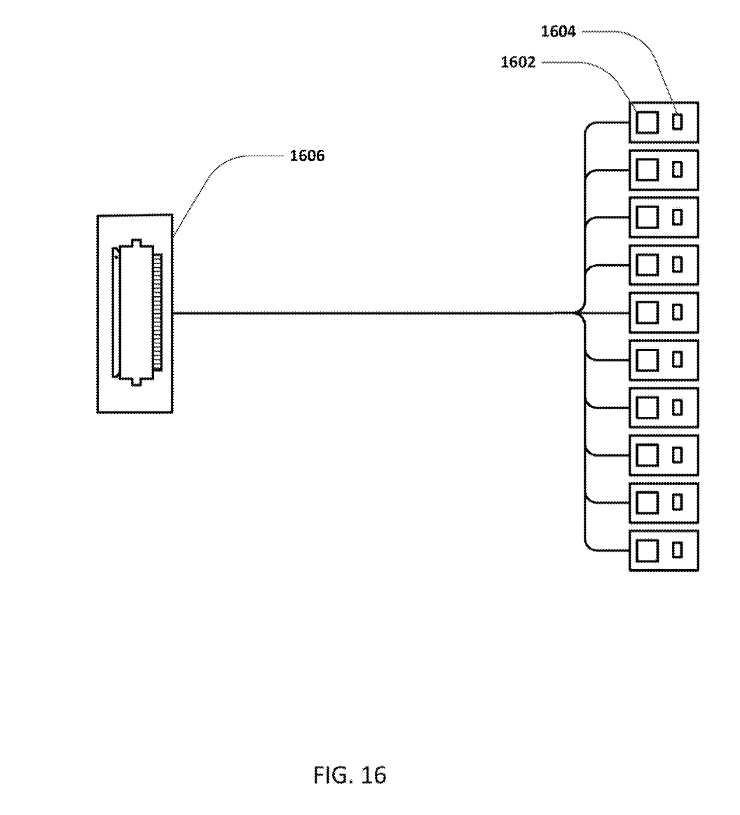

FIG. 16 is a non-limiting embodiment of at least a portion of the JVP monitoring system. In this embodiment, accelerometers 1602 and indicator lights 1604 are both located on the tabs. This allows the lights to be clearly visible on the neck of the patient which also allowing circuit board 1605 to be reduced in size. Indicator lights 1604 are not necessary in any of the embodiments described herein, but are an added feature to aid the user.

Accurate placement of the tabs which include at least the accelerometers is important to get an accurate reading. The tabs, or the entire device, may have an adhesive backing. The device may be packaged with a protective layer which protects the adhesive, similar to the protective layer of an adhesive bandage. The protective layer may be thin and flexible so that part of it may be peeled back to expose the adhesive backing of part of the tabs. This part of the tabs may then be adhered to the neck of the patient while the remainder of the protective later remains and holds the tabs in place with respect to each other. Once part of the tabs are adhered to the neck, the rest of the protective layer may be removed and the tabs firmly adhered to the neck. In this way, the tabs are easily adhered to the neck without losing their alignment with respect to each other. They can also operate independently of each other.

In the foregoing specification, the invention has been described with reference to specific embodiments thereof, and has been described as effective in providing methods and devices for estimating central venous pressure. However, it will be evident that various modifications and changes can be made thereto without departing from the broader spirit or scope of the invention as set forth in the appended claims. Accordingly, the specification is to be regarded in an illustrative rather than a restrictive sense.

The present invention may suitably comprise, consist or consist essentially of the elements disclosed and may be practiced in the absence of an element not disclosed. For instance, the pressure device configured to estimate central venous pressure may consist of or consist essentially at least one signal processor, at least one accelerometer, at least one memory for storing computer instructions related to the processor(s) and/or accelerometer(s), at least one display, and at least one patch adapted to be held in place or otherwise secured to a patient's neck; and the signal processor may be in communication with the accelerometer(s) to translate the output from the accelerometer(s) to yield a signal and calculate the central venous pressure.

The method for determining at least one central venous pressure may consist of or consist essentially of monitoring at least one signal with at least one accelerometer placed on a patient's neck where the accelerometer(s) is in communication with a processor, the signal(s) may correlate to pulse amplitude and/or angles of the patient at a particular position; and at least one central venous pressure may be computed by processing the signal(s) received from the accelerometer(s), and the central venous pressure may be displayed. Pulse slope of the signal(s) may also be used.

Although accelerometers are mentioned in several embodiments herein, other sensors may be used to detect skin movement above and/or blood movement in a blood vessel. For example, acoustic sensor(s), pressure sensor(s), ultrasound sensor(s), passive light sensors, visible or other wavelength light, microphone(s) may be used etc. Frequencies of skin and/or blood movement measured may range from 1-800 Hz or 800-20,000 Hz, or above 20,000 Hz.

In addition, although several embodiments herein mention using the JVP using the internal jugular vein, other blood vessels may be used.

In addition, similar technology may be used to determine other physiological parameters. For example, several embodiments described herein include removing the waveform data relating to the carotid pulses. This carotid data may be used to evaluate hemodynamic nuances specific to certain cardiac pathology (such as aortic stenosis, aortic regurgitation, hypertrophic cardiomyopathy, sub aortic stenosis). For example, waveform amplitude, slope, frequency, and other data may be used to evaluate the health of the patient based on the carotid or other pulses.

As an illustration, aortic stenosis is usually characterized by severe calcification and hardening of the aortic valve leaflets, such that blood has a difficult time ejecting from the left ventricle. This condition is usually first detected by a physician auscultating a “murmur” over the heart. However, a notable physical exam finding that is only present in severe aortic stenosis is a finding that is detected by skilled physicians known as “delayed carotid upstroke signal”—the physician places fingers over one carotid artery and can subjectively feel a delay in the carotid ejection. The carotid arteries have a delayed “upstroke” because the blood coming out of the left ventricle experiences a slight “pause” as it waits for the calcified aortic valve leaflets to open.

However, in most cases when a physician is not skilled enough to differentiate mild aortic stenosis from severe aortic stenosis (both have a similar murmur on physical exam), an expensive echocardiogram is performed instead to help determine the severity. The JVP monitoring system could, if placed on the carotid artery, detect the carotid artery upstroke signal and gate it to the ECG signal to be able to tell the physician whether the carotid upstroke is “delayed” in a very specific manner and one which can be quantified as opposed to the subjective manner in which it is done on the physical exam.

The analysis of the carotid signal may have value in detecting severe aortic stenosis in places where echocardiograms are either not readily available or affordable. If a physician hears a murmur that is suspicious for aortic stenosis, the JVP monitoring system could detect if a threshold time of “delayed carotid upstroke” is met, and if so, only those patients receive the expensive echocardiogram. Otherwise, they are relegated to another follow-up appointment a year or so later when the device can check for their carotid delay again.

The carotid artery signal may also be useful in detecting other cardiac conditions, such as hypertrophic cardiomyopathy, sub-aortic stenosis, and heart failure. It is also possible to quantify the “area under the curve” of the carotid artery pulsation in various phases of the respiratory cycle in order to achieve a stroke-volume variation between respirations so that a cardiac output estimate can be made.

In addition, similar technology can be used to monitor heart function and/or fluid status through venous pressure. The venous pressure can be accessed at the brachiocephalic vein (or other vein on an extremity) and the pressure can be measured at that location in a manner similar to those disclosed herein. Using a single reading and/or multiple readings, an increase or decrease in pressure can be measured. This can be accomplished by adding a pressure sensing element in line with the needle used to draw blood during the clinic visit. Since blood is routinely drawn during clinic visits, this would not require an additional puncture. The device could also be a standalone device and could be utilized in a home healthcare setting, clinic or hospital in an intermittent or continuous manner. This type of device could also be used for generally monitoring central venous pressure (CVP) in a much less invasive manner.

The words “comprising” and “comprises” as used throughout the claims, are to be interpreted to mean “including but not limited to” and “includes but not limited to”, respectively.

What is claimed is:

1. An apparatus for monitoring venous pressure, comprising:
one or more accelerometers each mounted upon one or more corresponding tabs which are positionable upon a skin of a patient and in proximity to an underlying vessel, wherein each tab is in a fixed arrangement with respect to one another and each tab is independently movable relative to an adjacent tab;
a platform in communication with the one or more accelerometers; and
a processor in communication with each of the one or more accelerometers, wherein the processor is programmed to monitor a position of a pulse height within the underlying vessel via movement of skin with the one or more accelerometers and determine a corresponding venous pressure.

2. The apparatus of claim 1 wherein the platform is configured for placement upon the skin and is comprised of a flexible material which conforms to contours of a neck of the patient.

3. The apparatus of claim 1 wherein the tabs are configured for placement upon the skin of a neck of the patient in proximity to an internal jugular vein.

4. The apparatus of claim 1 wherein the platform comprises a circuit board and the one or more corresponding tabs are isolated from one another via spaces or cutouts.

5. The apparatus of claim 1 wherein the one or more tabs are attached to the platform via corresponding necks or wires each having a flexibility which is sufficient to allow each accelerometer to move independently relative to one another.

6. The apparatus of claim 1 wherein the one or more accelerometers comprise a plurality of accelerometers.

7. The apparatus of claim 6 wherein the plurality of accelerometers are linearly aligned.

8. The apparatus of claim 1 further comprising an ECG sensor in communication with the processor, wherein the ECG sensor is configured to synchronize information from the one or more accelerometers with corresponding electrical activity of the heart.

9. The apparatus of claim 1 further comprising one or more light sources in proximity to the skin of the patient.

10. The apparatus of claim 1 wherein the processor is in communication with a display for viewing the venous pressure.

11. The apparatus of claim 1 wherein the processor is further programmed to subtract a waveform corresponding to an arterial pressure from the pulse height when determining the venous pressure.

12. The apparatus of claim 1 wherein the processor is further programmed to monitor for a change in amplitude between pulses and to determine a decay in amplitude in determining the venous pressure.

13. The apparatus of claim 1 wherein the processor is further programmed to determine a highest position along the underlying vessel based on an angle measured from a sternal angle and a body of the sternum via the one or more accelerometers in determining the venous pressure.

14. The apparatus of claim 1 wherein the processor is further programmed to determine a hemodynamic condition of the patient relating to cardiac pathology.

15. The apparatus of claim 1 wherein the venous pressure is a jugular venous pressure.

16. The apparatus of claim 1 wherein the venous pressure is a central venous pressure.

17. A method of determining venous pressure, comprising:
positioning one or more accelerometers mounted upon one or more corresponding tabs upon a skin of a patient and in proximity to an underlying vessel;
sensing movement of the skin via the one or more accelerometers, wherein each tab is in a fixed arrangement with respect to one another and each tab is independently movable relative to an adjacent tab;
determining a pulse height corresponding to the movement sensed by the one or more accelerometers via a processor in communication with each of the one or more accelerometers; and
calculating venous pressure corresponding to the pulse height.

18. The method of claim 17 wherein positioning one or more accelerometers further comprises positioning a platform in communication with the one or more accelerometers upon a neck of the patient in proximity to an internal jugular vein.

19. The method of claim 17 wherein sensing movement comprises isolating a movement of each tab from an adjacent tab.

20. The method of claim 17 wherein sensing movement comprises sensing movement of the skin via a plurality of accelerometers aligned linearly relative to one another.

21. The method of claim 17 further comprising synchronizing information from the one or more accelerometers with corresponding electrical activity of the heart via an ECG sensor in communication with the processor.

22. The method of claim 17 further comprising illuminating the skin via one or more light sources in proximity to the skin of the patient.

23. The method of claim 17 further comprising displaying the venous pressure upon a display in communication with the processor.

24. The method of claim 17 wherein determining a pulse height further comprises subtracting a waveform corresponding to an arterial pressure from the pulse height.

25. The method of claim 17 wherein determining a pulse height further comprises monitoring for a change in amplitude between pulses and determining a decay in amplitude in determining the venous pressure.

26. The method of claim 17 wherein determining a pulse height further comprises determining a highest position along the underlying vessel based on an angle measured from a sternal angle and a body of the sternum via the one or more accelerometers.

27. The method of claim 17 further comprising determining a hemodynamic condition of the patient relating to cardiac pathology.

28. The method of claim 17 wherein calculating venous pressure comprises calculating a jugular venous pressure.

29. The method of claim 17 wherein calculating venous pressure comprises calculating a central venous pressure.

30. The method of claim 17 further comprising re-positioning the one or more accelerometers to a second position upon the skin until a waveform is no longer sensed.

31. The method of claim 17 wherein calculating venous pressure comprises calculating a mean central venous pressure via a formula:
P=5+d·sin θ
where,
d=a distance from a sternal angle to a highest position that yields a waveform,
θ=an inclined angle of an upper body of the patient relative to a horizontal position.
